(19) United States
(12) Patent Application Publication
(10) Pub. No.: US 2009/0281502 A1
(43) Pub. Date: Nov. 12, 2009

(54) INFUSION SITE RETAINER

(76) Inventor: Robert Heitkamp, Mantador, ND (US)

Correspondence Address: DICKE, BILLIG & CZAJA, FIFTH STREET TOWERS, 100 SOUTH FIFTH STREET, SUITE 2250, MINNEAPOLIS, MN, 55402, US

(21) Appl. No.: 12/432,968

(22) Filed: Apr. 30, 2009

Related U.S. Application Data
(60) Provisional application No. 61/051,736, filed on May 9, 2008.

(51) Int. Cl. A61M 39/08

(52) U.S. Cl. 604/179

(57) ABSTRACT

An infusion site retainer configured to maintain tubing coupled to an infusion port includes a support configured to be secured to a patient and having a patient surface opposite a first surface, at least one guide configured to couple the tubing to the support and permit a first portion of the tubing to slide relative to the support, and a plurality of tubing stops configured to secure a second portion of the tubing in a substantially immobile relationship relative to the support.

### CROSS-REFERENCE TO RELATED APPLICATION

This Non-Provisional patent application claims the benefit of the filing date of U.S. Provisional Patent Application Ser. No. 61/051,736 filed May 9, 2008, entitled “INFUSION SITE RETAINER.”

### BACKGROUND

Intravenous infusion is the introduction of a liquid, such as a saline solution, into a vein of a patient for the therapeutic treatment of the patient. The intravenous infusion is introduced to a body site through an infusion port that generally has a needle suited for accessing the vein and tubing extending from the infusion port that communicates with a fluid receptacle, such as a sealed plastic bag.

Intravenous infusion is a common medical practice that is done in outpatient procedures, inpatient and other surgical procedures, and long-term care procedures. Some patients are sedated and move in an involuntary manner that has the potential to disrupt or remove the infusion port from the infusion site. Other patients are uncomfortable with the notion of intravenous infusion and are fearful that their voluntary movements might displace the infusion port. Still other patients are confused or agitated and pull at the tubing connected to the infusion port.

For therapeutic and other reasons, it is desirable to provide improved healthcare outcomes for patients undergoing intravenous infusion.

### SUMMARY

One aspect provides an infusion site retainer configured to maintain tubing coupled to an infusion port. The infusion site retainer includes a support configured to be secured to a patient and having a patient surface opposite a first surface, at least one guide configured to couple the tubing to the support and permit a first portion of the tubing to slide relative to the support, and a plurality of tubing stops configured to secure a second portion of the tubing in a substantially immobile relationship relative to the support.

### BRIEF DESCRIPTION OF THE DRAWINGS

The accompanying drawings are included to provide a further understanding of the disclosure and are incorporated in as a part of this specification. The drawings illustrate example embodiments and together with the description serve to explain principles of the invention. Other embodiments and many of the intended advantages of the embodiments will be readily appreciated as they become better understood by reference to the following detailed description. The elements of the drawings are not necessarily to scale relative to each other. Like reference numerals designate corresponding similar parts.

FIG. 1 is a perspective view of a first or upper surface of an infusion site retainer according to one embodiment.

FIG. 2 is a perspective view of a patient surface of the infusion site retainer shown in FIG. 1.

FIG. 3 is a top view of the first or upper surface of the infusion site retainer shown in FIG. 1.

FIG. 4 is a side view of the infusion site retainer shown in FIG. 1.

FIG. 5 is a proximal end view of the infusion site retainer shown in FIG. 1.

FIG. 6A is a cross-sectional view of a tubing guide of the infusion site retainer shown in FIG. 3.

FIG. 6B is a cross-sectional view of a tubing stop of the infusion site retainer shown in FIG. 3.

FIG. 7 is a top view of the infusion site retainer shown in FIG. 3 including attachment straps according to one embodiment.

FIG. 8 is a top view of the infusion site retainer shown in FIG. 7 employed to retain an infusion port and infusion tubing according to one embodiment.

### DETAILED DESCRIPTION

In the following Detailed Description, reference is made to the accompanying drawings which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments of the present invention can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

Embodiments provide an apparatus and method for securing tubing coupled to an infusion port that is insertable into a patient. The apparatus/method include attaching a support to the patient, retaining the tubing in a guide that is configured to allow the tubing to move relative to the support, and securing the tubing with a multiplicity of stops that are configured to hold the tubing substantially stationary relative to the support.

One embodiment provides an infusion site retainer including a support having a patient surface opposite a first surface, one or more guide(s) attached to the first surface of the support, multiple stops attached to the first surface of the support, and some means for securing the patient surface of the support to the patient. The support defines an opening configured to enable insertion/maintenance of an infusion port and its associated. The guide(s) is/are configured to retain infusion tubing in a sliding but controlled relationship relative to the support. The tubing stop(s) is/are configured to hold the infusion tubing in a substantially immobile relationship relative to the support to prevent forces applied to the tubing from disturbing the infusion port and creating discomfort for the patient.

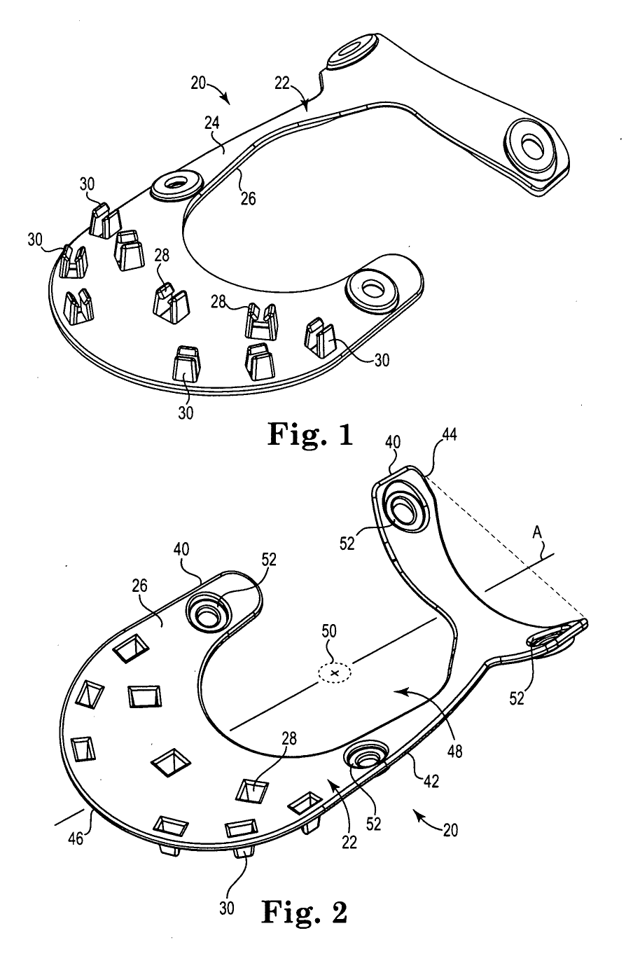

FIG. 1 is a perspective view of a first 24 or upper surface 24 of an infusion site retainer 20 configured to maintain tubing coupled to an infusion port according to one embodiment. Infusion site retainer 20 includes a support 22 with first surface 24 opposite a patient surface 26, and at least one guide 28 and tubing stops 30 attached to first surface 24.

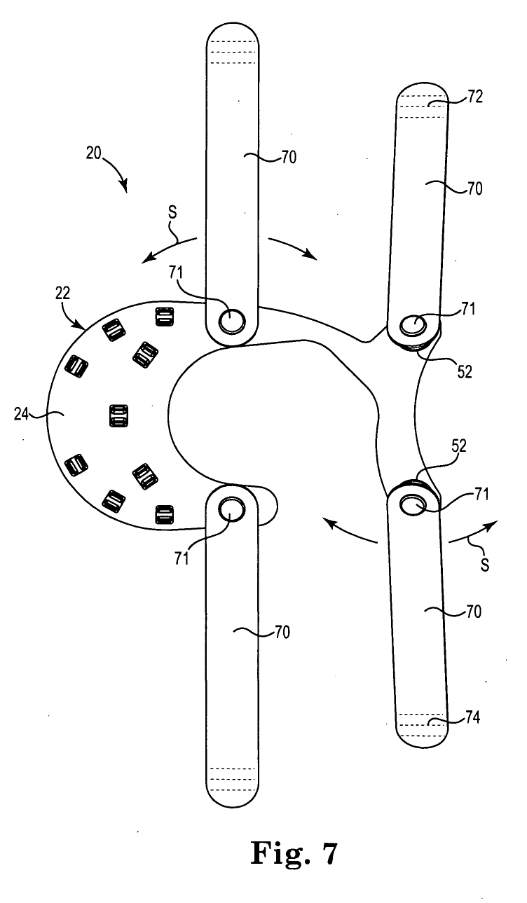

Support 22 is attachable to a patient. In one embodiment, support 22 is provided as a stand-alone component that is attached to the limb of the patient in any manner deemed suitable by the healthcare provider. For example, in one embodiment infusion site retainer 20 is configured to be taped or wrapped onto the limb of the patient. In one embodiment, infusion site retainer 20 includes straps (see FIG. 7) for attaching support 22 to the patient.

In one embodiment, support 22 is integrally formed from plastic to include guide(s) 28 and stops 30. One suitable plastic includes polypropylene. Other suitable plastics include polyethylene, or plastics having colorants, performance additives, anti-static additives, stabilizers or other constituents. In one embodiment, a fabric is attached to patient surface 26 of support 22 to provide the wearer with increased comfort.

In one embodiment, patient surface 26 includes an adhesive configured to attach support 22 to the patient. Suitable adhesives include acrylates in general, adhesive gels, water-retaining gel adhesives, or any other suitable adhesive configured for contact with patient skin. In one embodiment, only a portion of patient surface 26 of support 22 is provided with adhesive such that a suitable attachment is obtained against the patient's skin without attaching support 22 so tight to the patient's skin as to impede removal of support 22 after the infusion procedure.

Guide(s) 28 are configured to couple the infusion tubing to support 22 and permit a first portion of the tubing to slide relative to support 22. Tubing stops 30 are configured to secure a second portion of the tubing in a substantially immobile relationship relative to support 22. In one embodiment, support 22, guide(s) 28, and stops 30 are integrally formed as a single monolithic component.

The infusion site retainer 20 is attached to the patient by a healthcare worker in a suitable clean or antiseptic manner before the healthcare worker introduces an infusion port to the patient at the infusion site. The infusion port includes a needle that is introduced to a vein (or broadly, to the circulatory system of the patient). Infusion tubing extends from the infusion port to an infusion reservoir, which is typically a plastic bag containing infusion liquid.

Guide 28 of support 22 is configured to enable the infusion tubing to slide within guide 28, and thus comfortably move with local movement of the patient. Tubing stops 30 combine to secure or fix the infusion tubing in position relative to a periphery of support 22, such that a pull or a tug directed to the tubing will not transmit to the infusion port (that is to say, support 22 is configured such that tugs applied to the tubing are not transmitted to the infusion site). Since support 22 is attached to the patient, tubing stop 30 limits movement of the tubing relative to the patient. In this manner, the forces applied to the infusion tubing are buffered by support 22 in a manner that protects and retains the inserted infusion port even in the presence of significant forces applied to the tubing, which arise for example by the tubing getting caught on bedding, by the nursing staff inadvertently tugging on the tubing, or by voluntary and/or involuntary patient movements.

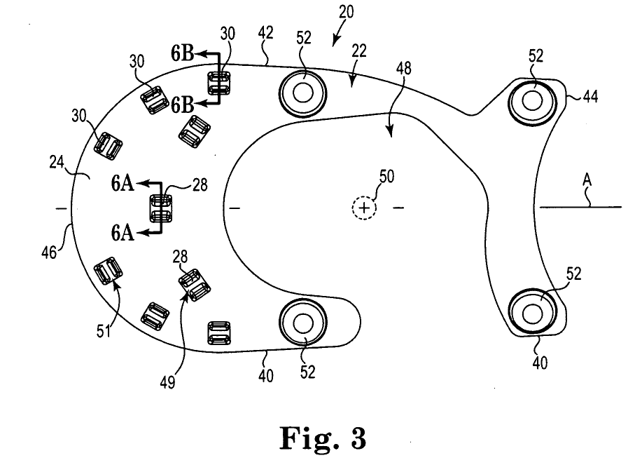

FIG. 2 is a perspective view of patient surface 26 of infusion site retainer 20 and FIG. 3 is a top view of patient surface 24 of infusion site retainer 20.

Support 22 includes opposing first and second lateral sides 40, 42 a proximal end 44 opposite a distal end 46, and an opening 48 formed in support 22 between proximal end 44 and distal end 46. In one embodiment, opening 48 is formed in one of the opposing first and second lateral sides 40, 42, for example side 40 as illustrated, to provide access to an infusion needle located in the opening 48 of support 22.

When support 22 is attached to a hand of the patient, proximal end 44 curves up, and distal end 46 curves around the rounded distal edge of support 22, to comfortably accommodate the wrist of the patient. In one embodiment, distal end 46 is smoothly curved and includes a bead slightly raised above the planar distal end that provides a comfort band/bead around a periphery of distal end 46.

In one embodiment, opening 48 is formed in support 22 between sides 40, 42 and ends 44, 46 and is sized to receive a needle or infusion port. In one embodiment, support 22 provides a length extending between ends 44, 46 of approximately 5.5 inches and a width extending between sides 40, 42 of about 3.5 inches. In one embodiment, opening 48 is an enclosed opening or a window having a peripheral boundary. In one embodiment, opening 48 is unbounded and is formed by removing a portion of side 40, as illustrated. The opening 48 may be circular, but is typically not circular. One suitable opening 48 is an unbounded opening having a length of about 3 inches and a width of about 2 inches, although other sizes for opening 48 are also acceptable.

In one embodiment, support 22 is a solid support that is not hollow. Suitable materials for forming support 22 include plastic and non-plastic. One suitable material includes high density polyethylene. Other suitable materials for forming support 22 include autoclavable plastics, recycled plastics, ethylene oxide sterilizable plastics, such as nylon or polyethylene, radiation sterilizable plastics such as polyethylene, moldable plastics or thermoformable plastics such as polypropylene. In one embodiment, support 22 is fabricated from a rigid cellulose material such as fiberboard or reinforced fiberboard.

With reference to FIG. 2, in one embodiment guides 28 and tubing stops 30 are integrally formed and molded with support 22 in a manner that forms breathing holes under each guide 28 (through channel 49) and support 30 (through channel 51), which allows air to flow through support 22 to the patient's skin. In one embodiment, support 22 includes grommets 52 illustrated in both FIGS. 2 and 3 that are configured to receive an attachment device suited for securing support 22 to the patient. In one embodiment, grommets 52 likewise form breathing holes through support 22 to allow air to flow to the patient's skin.

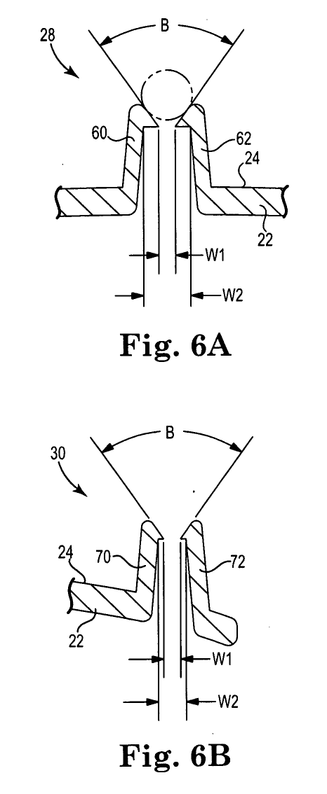

With reference to FIG. 3, in one embodiment guide 28 provides an open top channel 49 and tubing stop 30 provides an open top channel 51 that are both sized to receive infusion tubing. Channel 49 is sized to enable the infusion tubing to slide between the support walls of guide 28. For example, channel 49 is sized (see FIG. 6A) to enable the infusion tubing to be pressed into guide 28 in a manner that captures the infusion tubing, and once the tubing is pressed into guide 28, channel 49 allows the tubing to slide between the opposing walls of guide 28. In one embodiment, open top channel 51 of tubing stop 30 is similar in shape to channel 49, however the support walls that define channel 51 are configured to clamp down upon or grab the infusion tubing within tubing stop 30, and in this manner prevent the infusion tubing from tugging on the infusion port (see FIG. 6B).

In one embodiment, multiple guides 28 and multiple tubing stops 30 are provided on first surface 24 of support 22. For example, it has been discovered that embodiments of infusion site device 20 will protectively support the tubing in a manner that resists a tug or a pull of approximately 2.5 pounds force to the tubing. Supplying support 22 with four or more tubing stops 30, with each tubing stop 30 configured to resist about 0.5 pounds force of pull, results in support 22 resisting a tug to the tubing of at least 2.0 pounds or more of force. It is desirable to provide enough guides 28 to gather the tubing into a comfortable arrangement relative to the patient. As few as one guide 28 is acceptable, although three or more guides 28 are also acceptable.

In one embodiment, opening 48 defines a center 50 or spatial centroid 50 located near the position that a healthcare work would inserting the infusion port. With additional reference to FIG. 3, channels 49 of guides 28 are radially aligned with center 50 and tubing stops 30 are not aligned with center 50. In one embodiment, channels 51 of stops 30 are substantially orthogonal relative to center 50 and not in a line with channels 49 of guides 28. In one embodiment, each of the plurality of guides 28 is aligned radially with the centroid 50, and no tubing stop 30 is aligned radially with one of the guides. In this manner, the infusion tubing moves easily between guides 28 to relieve tension directed to center 50 and the infusion tubing is held in place by tubing stops 30 and not movable relative to support 22.

In one embodiment, the guides 28 are provided between opening 48 and stops 30 on distal end 46.

In one embodiment, tubing stops 30 are provided between guides 28 and distal end 46 and along a periphery of support 22 in such a manner that channel 51 within tubing stop 30 is not aligned with channel 49 of guide 28. In one embodiment, tubing stops 30 are disposed along a substantially semi-circular boundary of distal end 46 and each of the plurality of tubing stops 30 is equidistantly spaced along the semi-circular boundary of distal end 46.

In one embodiment, support 22 defines a central axis A having one guide 28 aligned on central axis A and aligned with center 50, with no tubing stop 30 disposed on central axis A. Each guide 28 is substantially orthogonal to the nearest tubing stop 30.

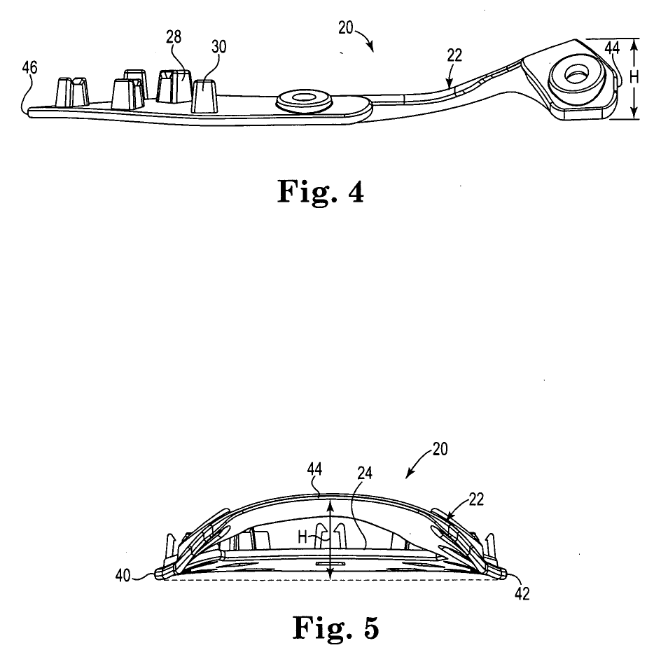

FIG. 4 is a side view of infusion site retainer 20. Support 22 forms a compound curvature extending from generally planar distal end 46 of support 22 to arched proximal end 44. In one embodiment, the compound curvature of support 22 is characterized by proximal end 44 of support 22 arching by a distance H out of the plane defined by distal end 46. In one embodiment, the distance H is molded to be between approximately 0.5 inches to 1.5 inches. In this manner, arched proximal end 44 rises up to accommodate movement of the patient's wrist when support 22 is secured to the patient. Guide(s) 28 are integrally formed with support 22 and tubing stops 30 are disposed on a periphery of support 22.

FIG. 5 is a view of proximal end 44 of infusion site retainer 20. Proximal end 44 is arched by the distance H to comfortably fit over the wrist of most if not all patients. Distal end 46 is configured to contact a back of the patient's hand, for example, and is generally planar. Support 22 has a compound curvature in which a portion of proximal end 44 including a portion of opening 48 is raised off of substantially planar distal end 46.

FIG. 6A is a cross-sectional view of tubing guide 28. Each guide 28 includes a first post 60 spaced apart from a second post 62. Each post 60, 62 tapers toward the other (e.g., is displaced from vertical) by an amount between approximately 5-15 degrees, preferably by about 10 degrees. The top end of each post 60, 62 is chamfered to provide an entrance angle B between approximately 60-80 degrees, where the entrance angle B configures posts 60, 62 to receive infusion tubing. The view of FIG. 6A includes an exemplary depiction of 0.180 inch diameter infusion tubing positioned for entrance into posts 60, 62. An entrance distance W1 is provided between the top ends of posts 60, 62, where the entrance distance W1 is selected to allow the infusion tubing to be inserted into guide 28 and remain positioned therein. A post offset distance W2 is provided between posts 60, 62 as measured adjacent to the top ends. The post offset distance W2 is generally larger than the entrance distance W1. Since the posts 60, 62 taper, the distance between posts 60, 62 as measured along first surface 24 of support 22 is greater than both the post offset distance W2 and the entrance distance W1. For guide 28, the post offset distance W2 is selected to allow the infusion tubing to slide between posts 60, 62.

In one embodiment, the entrance distance W1 is between approximately 0.050-0.070 inches, and is preferably about 0.060 inches; the post offset distance W2 is between approximately 0.150-0.180 inches, and is preferably about 0.166 inches.

In one embodiment, an inside height for posts 60, 62 is selected to accommodate various different diameter sizes of infusion tubing. For example, it is desirable that infusion tubing having a diameter of 0.180 inches slide through guide 28, and one embodiment for the inside height of posts 60, 62 is between approximately 0.1 to 0.5 inches to accommodate this diameter and allow a wide range of tubing having varying different diameters to slide between posts 60, 62.

FIG. 6B is a cross-sectional view of tubing stop 30. Each stop 30 includes a first post 70 spaced apart from a second post 72. Each post 70, 72 tapers toward the other (e.g., is displaced from vertical) by an amount between approximately 5-15 degrees, preferably by about 10 degrees. The top end of each post 70, 72 is chamfered to provide an entrance angle B between approximately 60-80 degrees, where the entrance angle B configures posts 70, 72 to receive infusion tubing. An entrance distance W1 is provided between the top ends of posts 70, 72, where the entrance distance W1 is selected to allow exemplary 0.180 inch diameter infusion tubing to be inserted into stop 30 and remain positioned therein. A post offset distance W2 is provided between posts 70, 72 as measured adjacent to the top ends. The post offset distance W2 is generally larger than the entrance distance W1. Since the posts 70, 72 taper, the distance between posts 70, 72 as measured along first surface 24 of support 22 is greater than both the post offset distance W2 and the entrance distance W1. For stop 30, the post offset distance W2 is selected to ensure that posts 70, 72 frictionally resist the sliding of the 0.180 inch diameter infusion tubing relative to tubing stop 30.

In one embodiment, the entrance distance W1 is between approximately 0.050-0.070 inches, and is preferably about 0.060 inches; the post offset distance W2 is between approximately 0.095-0.105 inches, and is preferably about 0.099 inches. In this manner, when the infusion tubing is captured between more than one of the stops 30, the stops 30 combine to secure the infusion tubing to prevent the tubing from tugging the infusion port, as further described below.

The above-identified dimensions are offered for exemplary purposes, and it should be understood that other dimensions may be suitably employed depending upon the tubing diameter. That is to say, the present application is not to be limited by the exemplary dimensions employed to describe the relative exemplary sizes of guides 28 and stops 30.

FIG. 7 is a view of top surface 24 of infusion site retainer 20 including attachment straps 70 according to one embodiment. Straps 70 attach to grommets 52 of support 22 and are configured to removably retain support 22 on the patient. In one embodiment, strap 70 is attached through grommet 52 with a swivel pin 71 such that strap 70 swivels relative to support 22 through an arc S. In this manner, strap 70 moves sufficiently to accommodate attachment around wrists having different diameters and differently sized hands, which enables straps 70 to comfortably accommodate differently sized patients.

Suitable straps 70 include tie straps, buckle straps, or straps with reclosable fasteners such as hook and loop fasteners. For example, in one embodiment first ends of one side of a hook-and-loop fastening strap 70 are provided with a hook portion 72 that mates with an opposing end of strap 70 having a loop portion 74 that enables strap 70 to be removably attachable around a limb of the patient.

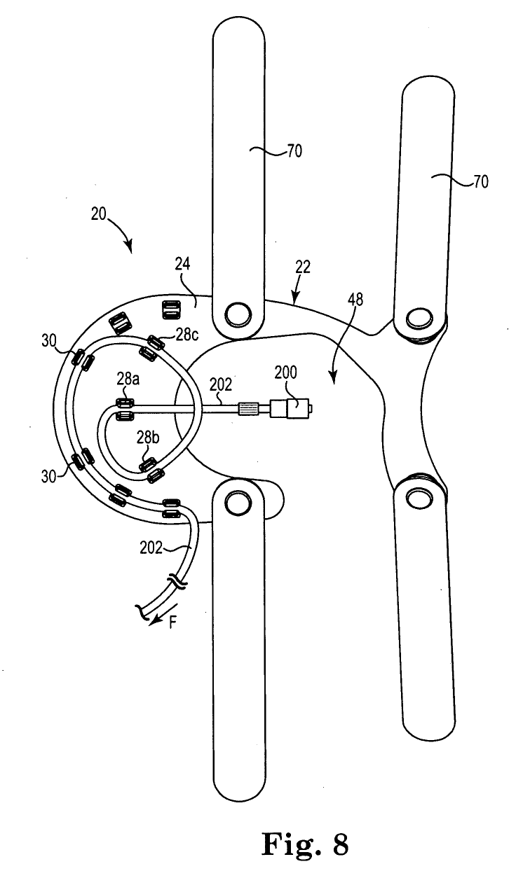

FIG. 8 is a view of top surface 24 of infusion site retainer 20 securing an infusion port 200 and infusion tubing 202 according to one embodiment. The infusion port 200 and the infusion site are disposed within a central portion of opening 48. The infusion tubing 202 extends from the infusion port 200 through center guide 28a into a left side guide 28b before wrapping around to a right side guide 28c and into tubing stops 30. Other pathways for tubing 202 relative to guides 28 and stops 30 are also acceptable.

Guides 28 enable a portion of the infusion tubing 202 nearest to infusion port 200 to slide relative to support 22, and thus comfortably move with the local movement of the patient. Tubing stops 30 (in this example four tubing stops 30) clamp or fix the infusion tubing 202 in position relative to a periphery of support 22, such that a pull or a tug having force F directed to an end portion of the tubing 202 nearest the infusion liquid reservoir will not transmit beyond the tubing stops 30 to the infusion port 200. The forces F applied to the infusion tubing 202 are buffered by the tubing stops 30 in a manner that protects and retains the inserted infusion port 200 even in the presence of significant forces applied to the tubing.

For example, it has been surprisingly discovered during an evaluation of infusion site retainer 20 that infusion port 200 remained inserted in the patient's vein even when tubing 202 unexpectedly became tangled around an ankle of a healthcare provider and tugged with substantial force. It is not uncommon for infusion tubing to become entangled in this or a similar manner in the active environment of a healthcare facility. However, the known forms of holding the infusion port in place (namely taping) lack the form and function to retain the infusion port in its inserted configuration when subjected to tugs and/or pulls. In contrast, infusion site retainer 20 resists the tugging and pulling of tubing 202 and protectively maintains port 200 in its desirably inserted location relative to the patient.

In addition, it has been determined during an evaluation of infusion site retainer 20 that the portion of the infusion tubing 202 nearest to infusion port 200 has a residual torque that causes the tubing/infusion port 200 to deflect laterally as the new tubing is removed from its packaging. This residual torque in the tubing has been evaluated to have a value between approximately 1.5-2.0 inch-ounces. It has been surprisingly discovered during an evaluation of infusion site retainer 20 that guides 28 and support 22 combine to relieve the residual torque in the tubing 202, which is expected to result in more comfort for the patient and improved retention of the infusion port 200.

Embodiments provide an infusion site retainer suited for use by animals or humans at peripheral, central, or other infusion sites. The above described infusion site retainers are configured to not limit the full range of motion for extremities such as a hand in a manner that safely retains the connection between the infusion tubing and the infusion port.

Embodiments of the infusion site retainers provide a low cost, disposable, or reusable solution suited for day surgery or hospital or long term care procedures.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific infusion site retainers discussed herein. Therefore, it is intended that this invention be limited only by the claims and the equivalents thereof.

What is claimed is:

1. An infusion site retainer configured to maintain tubing coupled to an infusion port, the infusion site retainer comprising:
a support configured to be secured to a patient and comprising a patient surface opposite a first surface;
at least one guide configured to couple the tubing to the support and permit a first portion of the tubing to slide relative to the support; and
a plurality of tubing stops configured to secure a second portion of the tubing in a substantially immobile relationship relative to the support.

2. The infusion site retainer of claim 1, wherein the at least one guide and the plurality of tubing stops are attached to the first surface of the support.

3. The infusion site retainer of claim 1, wherein the at least one guide and the plurality of tubing stops are integrally formed as a part of the first surface of the support.

4. The infusion site retainer of claim 3, wherein each of the plurality of tubing stops comprises a first post spaced apart from a second post with a distance between the first and second posts measured along the first surface of the support being greater than a distance between the first and second posts measured at their ends.

5. The infusion site retainer of claim 1, wherein the support defines an opening sized for placement around a perimeter of the infusion port.

6. The infusion site retainer of claim 5, wherein the opening is disposed between a distal end of the support and a proximal end of the support, the at least one guide is attached to the first surface of the support between the window and the distal end, and the plurality of tubing stops is attached to the first surface of the support between the at least one guide and the distal end.

7. The infusion site retainer of claim 6, wherein the support comprises opposing first and second lateral sides and the opening comprises an unbounded opening formed in a portion of one of the opposing first and second lateral sides.

8. The infusion site retainer of claim 6, wherein the distal end of the support is curved to define a substantially semi-circular boundary and each of the plurality of tubing stops is equidistantly spaced along the semi-circular boundary.

9. The infusion site retainer of claim 6, wherein the proximal end of the support is arched to define an arched curve in lateral cross-section, the arched curve configured for placement over a wrist of the patient.

10. The infusion site retainer of claim 1, wherein the plurality of tubing stops is attached to the first side of the support adjacent to a distal end of the support and comprising a plurality of guides attached to the first surface of the support between the plurality of tubing stops and a proximal end of the support.

11. The infusion site retainer of claim 10, wherein the support defines a centroid and each of the plurality of guides is aligned radially with the centroid.

12. The infusion site retainer of claim 11, wherein no tubing stop is aligned radially with one of the guides.

13. The infusion site retainer of claim 1, further comprising:
a strap configured to removably secure the support to a limb of the patient, the strap comprising a first end attached to the support by a swivel.

14. An infusion site retainer configured to maintain tubing coupled to an infusion port insertable into a patient, the infusion site retainer comprising:
a support comprising a patient surface opposite a first surface, the support extending between a proximal end and a distal end and defining an opening that is longer than the infusion port;
a guide attached to the first surface of the support between the opening and the distal end of the support, the guide aligned radially with a center of the opening and configured to enable the tubing to move relative to the support;
at least two tubing stops attached to the first surface of the support and disposed along a periphery of the distal end of the support, the tubing stops configured to hold the tubing substantially stationary relative to the support; and
an attachment mechanism attached to the support and configured for securing the patient surface of the support to the patient.

15. The infusion site retainer of claim 14, wherein the support comprises a J-shaped support comprising opposing first and second lateral sides extending between the distal end and the proximal end, and the opening is formed in a portion of one of the opposing first and second lateral sides.

16. The infusion site retainer of claim 14, wherein each of the tubing stops comprises a first post spaced apart from a second post, the first and second posts integrally formed with the first surface of the support such that ends of the posts converge toward each other.

17. A method securing tubing coupled to an infusion port insertable into a patient, the method comprising:
attaching a support to the patient;
retaining the tubing in at least one guide that is configured to allow the tubing to move relative to the support; and
securing the tubing to multiple stops that are configured to hold the tubing substantially stationary relative to the support.

18. The method of claim 17, comprising configuring the guide to allow the tubing to slide relative to the guide.

19. The method of claim 17, comprising enabling a patient-end of the tubing to slide within the guide and holding an opposing end of the tubing within the multiple stops.

20. The method of claim 17, comprising securing the tubing within 4 stops such that the tubing is held substantially stationary relative to the support under a tension of approximately 2.0 pounds force.
